(19) United States
(12) Patent Application Publication
(10) Pub. No.: US 2022/0226144 A1
(43) Pub. Date: Jul. 21, 2022

(54) FLUID COLLECTION DEVICES AND SYSTEMS HAVING A FLUID IMPERMEABLE BARRIER WITH A SELECTIVELY MINIMAL HARDNESS, THICKNESS, AND/OR MODULUS OF ELASTICITY

(71) Applicant: PureWick Corporation, El Cajon, CA (US)

(72) Inventors: Ashley Marie Johannes, Atlanta, GA (US); Pranav Challa, Atlanta, GA (US)

(21) Appl. No.: 17/595,747

(22) PCT Filed: Mar. 19, 2020
(86) PCT No.: PCT/US2020/023572

§ 371 Date: Nov. 23, 2021

Related U.S. Application Data
(60) Provisional application No. 62/853,889, filed on May 29, 2019.

(51) Int. Cl. A61F 5/455

(52) U.S. Cl. CPC A61F 5/455

(57) ABSTRACT

Example fluid collection devices and methods of assembling the fluid collection devices are described. The fluid collection devices include a fluid impermeable barrier and a fluid permeable body. The fluid impermeable barrier has a shore A durometer hardness of less than about (15). The fluid impermeable barrier at least partially defines a chamber and an opening extending therethrough that is configured to be positioned adjacent to a urethra of a subject. The fluid permeable body is positioned at least partially within the chamber to extend across at least a portion of the opening and configured to wick fluid away from the opening.

### CROSS-REFERENCE TO RELATED APPLICATIONS

This application claims priority to U.S. Provisional Application No. 62/853,889 filed on 29 May 2019, the disclosure of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, can be used to address some of these circumstances. However, bed pans and urinary catheters have several problems associated therewith. For example, bed pans can be prone to discomfort, pressure ulcers spills, and other hygiene issues. Urinary catheters be can be uncomfortable, painful, and can cause urinary tract infections.

Thus, users and manufacturers of fluid collection devices continue to seek new and improved devices, systems, and methods to collect urine.

### SUMMARY

Embodiments disclosed herein are fluid collection devices and methods of assembling fluid collection devices. In an embodiment, a fluid collection device includes a fluid impermeable barrier and a fluid permeable body. The fluid impermeable barrier has a shore A durometer hardness of less than about 15. The fluid impermeable barrier at least partially defines a chamber, an opening extending longitudinally along the fluid impermeable barrier and configured to be positioned adjacent to a female urethra, and an aperture configured to receive a conduit therethrough. The fluid permeable body is positioned at least partially within the chamber to extend across at least a portion of the opening and configured to wick fluid away from the opening.

In an embodiment, a fluid collection device includes a fluid impermeable barrier and a fluid permeable body. The fluid impermeable barrier has a thickness of about 1/64 inch (0.397 mm) to about ⅛ inch (3.175 mm). The fluid impermeable barrier at least partially defines a chamber, an opening extending longitudinally along the fluid impermeable barrier and configured to be positioned adjacent to a female urethra, and an aperture configured to receive a conduit therethrough. The fluid permeable body defining is positioned at least partially within the chamber to extend across at least a portion of the opening and configured to wick fluid away from the opening.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a female fluid collection device, according to an embodiment.

FIG. 1B is a front view of the female fluid collection device of FIG. 1A worn on a female user.

FIG. 1C is an exploded view of the female fluid collection device of FIG. 1A.

FIGS. 2A and 2B are cross-sectional views of the female fluid collection device of FIG. 1 taken along line 2-2 thereof, according to various embodiments.

FIG. 3 is a flow diagram of a method of assembling a fluid collection device, according to an embodiment.

FIG. 4 is a flow diagram of a method to collect fluid, according to an embodiment.

FIG. 5 is a block diagram of a system for fluid collection, according to an embodiment.

### DETAILED DESCRIPTION

Embodiments disclosed herein are fluid collection devices and methods of assembling fluid collection devices. Many conventional fluid collection devices include a hard, stiff, fluid impermeable outer layer that can injure the skin of the wearer when the fluid collection device is removed due to the friction between the skin and the fluid impermeable outer layer. Embodiments described herein may include a fluid impermeable layer having a lower hardness than the fluid impermeable outer layer of conventional fluid collection devices. For example, in an embodiment, a fluid collection device includes a fluid impermeable barrier and a fluid permeable body. The fluid impermeable barrier has a shore A durometer hardness of less than about 15. In many embodiments, the fluid impermeable barrier includes a lower hardness than fluid impermeable barriers of conventional fluid collection devices. The lower hardness of the fluid impermeable barriers described herein provides a softer, thinner material that is less stiff and more comfortable for the user to wear. A lower hardness fluid impermeable barrier also aids in securement of the fluid impermeable barrier to the skin of the user. The fluid impermeable barrier at least partially defines a chamber, an opening extending longitudinally along the fluid impermeable barrier and configured to be positioned adjacent to the opening of a female urethra, and an aperture configured to receive a conduit therethrough. The fluid permeable body is positioned at least partially within the chamber to extend across at least a portion of the opening and configured to wick fluid away from the opening.

In an embodiment, a fluid collection device includes a fluid impermeable barrier and a fluid permeable body. The fluid impermeable barrier has a thickness of about 0.008 inch (0.2 mm) to about 0.125 inch (3.2 mm). The fluid impermeable barrier at least partially defines a chamber, an opening extending longitudinally along the fluid impermeable barrier and configured to be positioned adjacent to a female urethra, and an aperture configured to receive a conduit therethrough. The fluid impermeable barrier of many embodiments is thinner than fluid impermeable barriers of conventional fluid collection devices. The relative thinness of the fluid impermeable barrier provides a more comfortable fluid collection device for the user to wear. The fluid permeable body is positioned at least partially within the chamber to extend across at least a portion of the opening and configured to wick fluid away from the opening.

The fluid collection devices disclosed herein are configured to collect fluids from an individual. The fluids collected by the fluid collection devices may include urine. The fluids collected by the fluid collection devices may also include at least one of vaginal discharge, penile discharge, reproductive fluids, blood, sweat, or other bodily fluids.

Fluid collection devices described herein may be used in fluid collection systems. The fluid collection systems may include a fluid collection device, a fluid storage container, and a portable vacuum source. Fluid (e.g., urine or other bodily fluids) collected in the fluid collection device may be removed from the fluid collection device via a conduit which protrudes into an interior region of the fluid collection device. For example, a first open end of the conduit may extend into the fluid collection device to a reservoir therein. The second open end of the conduit may extend into the fluid collection device or the portable vacuum source. The suction force may be introduced into the interior region of the fluid collection device via the first open end of the conduit responsive to a suction (e.g., vacuum) force applied at the second end of the conduit. The suction force may be applied to the second open end of the conduit by the portable vacuum source either directly or indirectly.

Fluid collection devices described herein may be shaped and sized to be positioned adjacent to the opening of a female urethra or have a male urethra positioned therethrough (e.g., receive a penis therein). For example, the fluid collection device may include a fluid impermeable barrier at least partially defining a chamber (e.g., interior region of the fluid collection device) of the fluid collection device. The fluid impermeable barrier also defines an opening extending therethrough from the external environment. The opening may be positioned adjacent to a female urethra or have a male urethra positioned therethrough. The fluid collection device may include a fluid permeable body disposed within the fluid impermeable barrier. The conduit may extend into the fluid collection device at a first end region, through one or more of the fluid impermeable barrier, fluid permeable body to a second end region of the fluid collection device. Exemplary fluid collection devices for use with the systems and methods herein are described in more detail below.

In some embodiments, the portable vacuum source may be disposed in or on the fluid collection device. In such embodiments, the conduit may extend from the fluid collection device and attach to the portable vacuum source at a first point therein. An additional conduit may attach to the portable vacuum source at a second point thereon and may extend out of the fluid collection device, and may attach to the fluid storage container. Accordingly, a vacuum (e.g., suction) may be drawn through the fluid collection device via the fluid storage container. Fluid, such as urine, may be drained from the fluid collection device using the portable vacuum source.

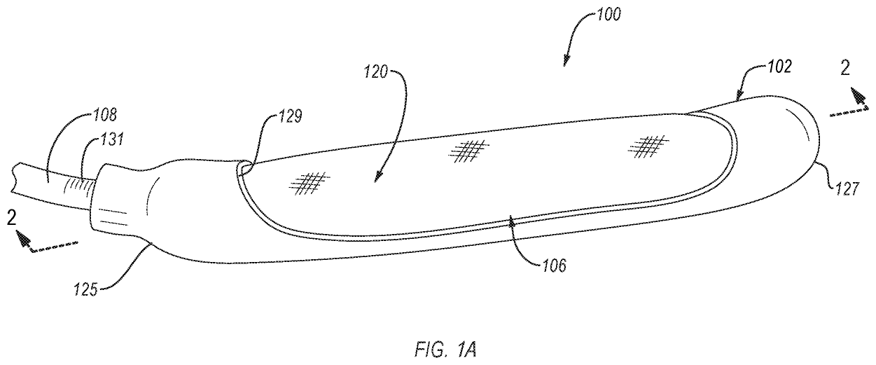

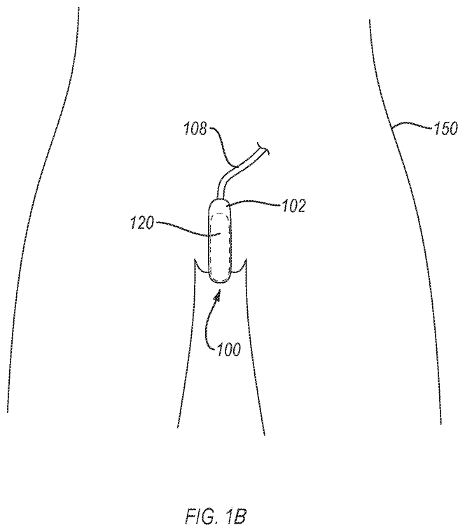

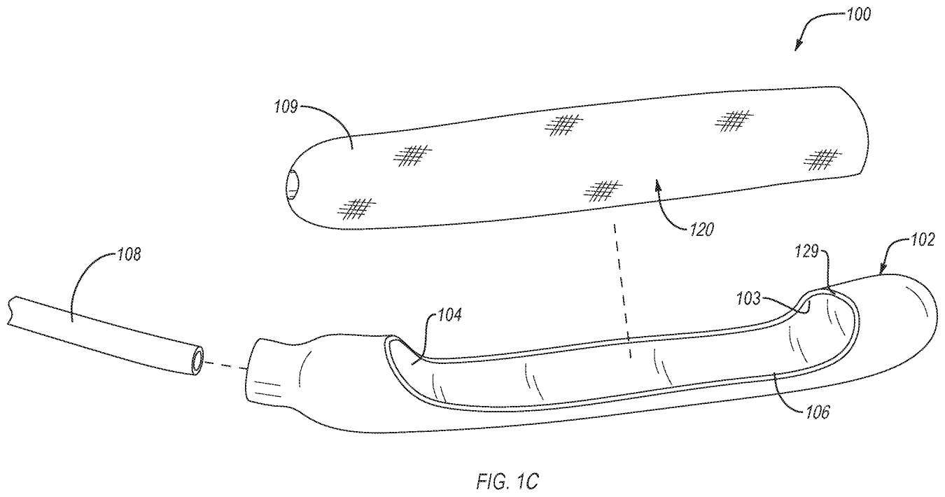

FIG. 1A is an isometric view of a fluid collection device 100, according to an embodiment. The fluid collection device 100 is an example of a female fluid collection device 100 that is configured to receive fluids from a female. The fluid collection device 100 includes a fluid impermeable barrier 102 having a first end region 125 and a second end region 127. The fluid impermeable barrier 102 at least partially defines a chamber 104 (e.g., interior region, shown in FIG. 1C) and includes an inward border or edge 129 defining an opening 106. The fluid impermeable barrier 102 is substantially cylindrical in shape between the first end region 125 and the second end region 127. In other embodiments, the fluid impermeable barrier 102 may include other shapes, such as one of more substantially planar surfaces, triangular, or other suitable shape. The opening 106 is formed in and extends longitudinally through the fluid impermeable barrier 102, thereby enabling fluids to enter the chamber 104 from outside of the fluid collection device 100. The opening 106 may be configured to be positioned adjacent to the opening of a female urethra.

The fluid collection device 100 may be positioned proximate to the opening of the female urethra and urine may enter the interior region of the fluid collection device 100 via the opening 106. The fluid collection device 100 is configured to receive the fluids into the chamber 104 via the opening 106. For example, the opening 106 may exhibit an elongated shape that is configured to extend from a first location below the urethral opening (e.g., at or near the anus or the vaginal opening) to a second location above the urethral opening (e.g., at or near the clitoris or the pubic hair). The opening 106 may exhibit an elongated shape since the space between the legs of a female is relatively small when the legs of the female are closed, thereby only permitting the flow of the fluids along a path that corresponds to the elongated shape of the opening 106. For example, the opening may extend longitudinally along the fluid impermeable barrier. The opening 106 in the fluid impermeable barrier 102 may exhibit a width that is measured transverse to the longitudinal direction and may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The opening 106 may exhibit a width that is greater than 50% of the circumference of the fluid collection device 100 since the vacuum (e.g., suction) through the conduit 108 pulls the fluid into the conduit 108. In some embodiments, the opening 106 may be vertically oriented (e.g., having a major axis parallel to the longitudinal axis of the device 100). In some embodiments, (not shown), the opening 106 may be horizontally oriented (e.g., having a major axis perpendicular to the longitudinal axis of the device 100). In some embodiments, the inward border or edge 129 of the fluid impermeable barrier 102 defines the opening 106. The edge 129 may include two opposing arced portions, the arc portions following the outer circumference or periphery of the substantially cylindrical fluid impermeable barrier 102. In an embodiment, the fluid impermeable barrier 102 may be configured to be attached to the individual, such as adhesively attached (e.g., with a hydrogel adhesive) to the individual. According to an embodiment, a suitable adhesive is a hydrogel layer, such as those disclosed in U.S. Patent Application Publication No. 2017/0189225, the disclosure of which is incorporated herein by reference in its entirety.

The fluid impermeable barrier 102 may also temporarily store the fluids in the chamber 104. For example, the fluid impermeable barrier 102 may be formed of any suitable fluid impermeable materials, such as a fluid impermeable polymer (e.g., silicone, polypropylene, polyethylene, polyethylene terephthalate, a polycarbonate, etc.), polyurethane films, thermoplastic elastomer (TPE), rubber, thermoplastic polyurethane, another suitable material, or combinations thereof. As such, the fluid impermeable barrier 102 substantially prevents the fluids from exiting the portions of the chamber 104 that are spaced from the opening 106. The fluid impermeable barrier 102 is flexible, thereby enabling the fluid collection device 100 to bend or curve when positioned against the body of a wearer. Example fluid impermeable barriers may include, but are not limited to, a fluid impermeable barrier including at least one of Versaflex CL 2000X TPE, Dynaflex G6713 TPE, or Silpuran 6000/05 A/B silicone.

In many embodiments, the fluid impermeable barrier 102 includes a lower hardness than fluid impermeable barriers of conventional fluid collection devices. Conventional fluid collection devices typically include a hard, stiff, fluid impermeable outer layer that may injure the skin of the wearer when the fluid collection device is removed due to the friction between the skin and the fluid impermeable outer layer. The lower hardness of the fluid impermeable barrier 102 provides a softer, thinner material that is less stiff and more comfortable for the user to wear. A lower hardness fluid impermeable barrier 102 also aids in securement of the fluid impermeable barrier to the skin of the user. The fluid impermeable barrier 102 including silicone, thermoplastic elastomer, rubber, thermoplastic polyurethane, or combinations thereof provides the lower hardness described herein. Hardness of the fluid impermeable barrier 102 may include one or more of a shore A durometer hardness or a shore 00 durometer hardness. A shore A durometer hardness of the fluid impermeable barrier 102 may vary according to different embodiments. For example, the fluid impermeable barrier 102 may include a shore A durometer hardness of about 0 to about 30, about 2 to about 25, about 3 to about 20, about 4 to about 15, about 0 to about 5, about 5 to about 10, about 10 to about 15, about 15 to about 20, about 20 to about 25, about 25 to about 30, about 1 to about 3, about 3 to about 5, about 5 to about 7, about 7 to about 9, about 9 to about 11, about 11 to about 13, about 13 to about 15, about 15 to about 17, about 17 to about 19, about 19 to about 21, about 21 to about 23, about 23 to about 25, about 25 to about 27, about 27 to about 29, about 2, about 4 about 6, about 8, about 10, about 12, about 14, about 16, about 18, about 20, about 22, about 24, about 26, about 28, about 30, less than about 30, less than about 27.5, less than about 25, less than about 22.5, less than about 20, less than about 17.5, less than about 15, less than about 12.5, less than about 10, less than about 8, less than about 7, less than about 6, less than about 5, less than about 4, less than about 3, less than about 2, less than about 1, or about 0. In some examples, the fluid impermeable barrier 102 may include Versaflex CL 2000X TPE having a shore A durometer hardness of about 3, Dynaflex G6713 TPE having a shore A durometer of about 14, or Silpuran 6000/05 AB silicone having a shore A durometer hardness of about 5.

A shore 00 durometer hardness of the fluid impermeable barrier 102 also may vary according to different embodiments. For example, the fluid impermeable barrier 102 may include a shore 00 durometer hardness of about 10 to about 70, about 15 to about 65, about 20 to about 60, about 25 to about 55, about 30 to about 50, about 35 to about 45, about 10 to about 15, about 15 to about 20, about 20 to about 25, about 25 to about 30, about 30 to about 35, about 30 to about 35, about 35 to about 40, about 40 to about 45, about 45 to about 50, about 50 to about 55, about 55 to about 60, about 60 to about 65, about 65 to about 70, less than about 70, less than about 65, less than about 60, less than about 55, less than about 50, less than about 45, less than about 40, less than about 35, less than about 30, less than about 25, less than about 20, or less than about 15.

In many embodiments, the fluid impermeable barrier 102 also may be constructed relatively thinner than fluid impermeable barriers of conventional fluid collection devices. The relative thinness of the fluid impermeable barrier 102 provides a more comfortable fluid collection device 100 for the user to wear. The thickness of the fluid impermeable barrier 102 may vary according to different embodiments. For example, the fluid impermeable barrier 102 may include a thickness of about 0.008 inch (0.203 mm) to about 0.125 inch (3.175 mm), about 0.016 inch (0.406 mm) to about 0.063 inch (1.600 mm), about 0.031 inch (0.787 mm) to about 0.125 inch (3.175 mm), about 0.008 inch (0.203 mm) to about 0.016 inch (0.406 mm), about 0.016 inch (0.406 mm) to about 0.031 inch (0.787 mm), about 0.031 inch (0.787 mm) to about 0.063 inch (1.600 mm), about 0.063 inch (1.600 mm) to about 0.125 inch (3.175 mm), about 0.008 inch (0.203 mm) to about 0.023 inch (0.584 mm), about 0.023 inch (0.584 mm) to about 0.031 inch (0.787 mm), about 0.031 inch (0.787 mm) to about 0.047 inch (1.193 mm), about 0.047 inch (1.193 mm) to about 0.063 inch (1.600 mm), about 0.063 inch (1.600 mm) to about 0.078 inch (1.981 mm), about 0.078 inch to about 0.094 inch (2.388 mm), about 0.094 inch (2.388 mm) to about 0.109 inch (2.769 mm), about 0.109 inch (2.769 mm) to about 0.125 inch (3.175 mm), about 0.008 inch (0.203 mm), about 0.016 inch (0.406 mm), about 0.031 inch (0.787 mm), about 0.047 inch (1.193 mm), about 0.063 inch (1.600 mm), about 0.078 inch (1.981 mm), about 0.094 inch (2.388 mm), about 0.109 inch (2.769 mm), about 0.125 inch (3.175 mm), less than about 0.125 inch (3.175 mm), less than about 0.109 inch (2.769 mm), less than about 0.094 inch (2.388 mm), less than about 0.078 inch (1.981 mm), less than about 0.063 inch (1.600 mm), less than about 0.047 inch (1.193 mm), less than about 0.031 inch (0.787 mm), less than about 0.016 inch (0.406 mm), or less than about 0.008 inch (0.203 mm).

In many embodiments, the fluid impermeable barrier 102 may include a lower modulus of elasticity than fluid impermeable barriers of conventional fluid collection devices. The modulus of elasticity of the fluid impermeable barrier 102 may vary according to different embodiments. For example, the fluid impermeable barrier 102 may include a modulus of elastic of about 1 N/in to about 30 N/in, about 2 N/in to about 26 N/in, about 3 N/in to about 23 N/in, about 4 N/in to about 20 N/in, about 5 N/in to about 10 N/in, about 1 N/in to about 5 N/in, about 6 N/in to about 10 N/in, about 11 N/in to about 15 N/in, about 16 N/in to about 20 N/in, about 21 N/in to about 25 N/in, about 26 N/in to about 30 N/in, about 2 N/in to about 4 N/in, about 4 N/in to about 6 N/in, about 6 N/in to about 8 N/in, about 8 N/in to about 10 N/in, about 10 N/in to about 12 N/in, about 12 N/in to about 14 N/in, about 14 N/in to about 16 N/in, about 16 N/in to about 18 N/in, about 18 N/in to about 20 N/in, about 20 N/in to about 22 N/in, about 22 N/in to about 24 N/in, about 24 N/in to about 26 N/in, about 26 N/in to about 28 N/in, about 28 to about 30 N/in, about 8 N/in, about 9 N/in, about 10 N/in, about 11 N/in, about 12 N/in, about 13 N/in, about 14 N/in, about 15 N/in, about 16 N/in, less than 30 N/in, less than about 25 N/in, less than about 20 N/in, less than about 18 N/in, less than about 16 N/in, less than about 16 N/in, less than about 14, N/in, less than about 12 N/in, less than about 10 N/in, less than about 8 N/in, less than about 6 N/in, less than about 4 N/in, or less than about 2 N/in.

In an embodiment, the fluid impermeable barrier 102 may be air permeable. In such an embodiment, the fluid impermeable barrier 102 may be formed of a hydrophobic material that defines a plurality of pores. In an embodiment, one or more portions of at least the outer surface of the fluid impermeable barrier 102 may be formed from a soft and/or smooth material, thereby reducing chaffing. The fluid impermeable barrier 102 may include markings thereon, such as one or more markings to aid a user in aligning the device 100 on the wearer. For example, a line on the fluid impermeable barrier 102 (e.g., opposite the opening 106) may allow a healthcare professional to align the opening 106 over the urethra of the wearer. In examples, the markings may include one or more of alignment guide or an orientation indicator, such as a stripe or hashes. Such markings may be positioned to align the device 100 to one or more anatomical features such as a pubic bone, etc.

The fluid collection device 100 may include a fluid permeable body 120 or layer disposed in the chamber 104. The fluid permeable body 120 may cover or extend across at least a portion (e.g., all) of the opening 106. The fluid permeable body 120 may be configured to wick any fluid away from the opening 106, thereby preventing the fluid from escaping the chamber 104. The fluid permeable body 120 also may wick the fluid generally towards an interior of the chamber 104, as discussed in more detail below. A portion of the fluid permeable body 120 may define a portion of an outer surface of the fluid collection device 100. Specifically, the portion of the fluid permeable body 120 defining the portion of the outer surface of the fluid collection device 100 may be the portion of the fluid permeable body 120 exposed by the opening 106 defined by the fluid impermeable barrier 102 that contacts the user. Moreover, the portion of the fluid permeable device defining the portion of the outer surface of the fluid collection device 100 may be free from coverage by gauze or other wicking material at the opening.

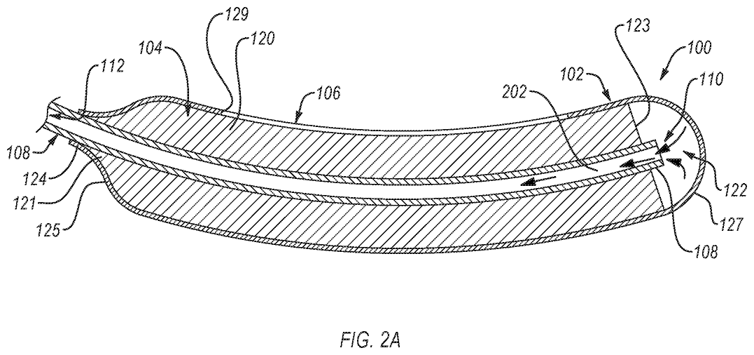

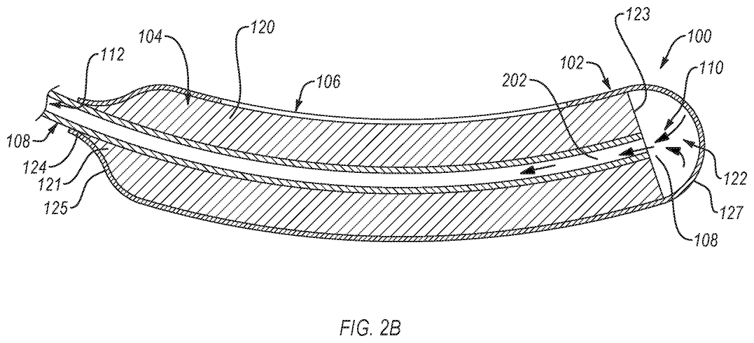

The fluid permeable body 120 may include any material that may wick the fluid. The permeable properties referred to herein may be wicking, capillary action, diffusion, or other similar properties or processes, and are referred to herein as “permeable” and/or “wicking.” Such “wicking” may exclude absorption into the wicking material. The fluid permeable body 120 may include a one-way fluid movement fabric. As such, the fluid permeable body 120 may remove fluid from the area around the female urethra, thereby leaving the urethra dry. The fluid permeable body 120 may enable the fluid to flow generally towards a reservoir 122 (shown in FIGS. 2A and 2B) of void space formed within the chamber 104. For example, the fluid permeable body 120 may include a porous or fibrous material, such as hydrophilic polyolefin. In some embodiments, the fluid permeable body 120 consists of or consists essentially of a porous or fibrous material, such as hydrophilic polyolefin. Examples of polyolefin that may be used in the fluid permeable body 120 include, but are not limited to, polyethylene, polypropylene, polyisobutylene, ethylene propylene rubber, ethylene propylene diene monomer, or combinations thereof. The porous or fibrous material may be extruded into a substantially cylindrically shape to fit within the chamber 104 of the fluid impermeable barrier 102. The fluid permeable body 120 may include varying densities or dimensions. Moreover, the fluid permeable body 120 may be manufactured according to various manufacturing methods, such as molding, extrusion, or sintering.

In some embodiments, the fluid permeable body 120 includes a singular and porous body. That is, during use, the fluid permeable body 120 extends from the conduit 108 to interface the fluid impermeable barrier 102 and the opening 106. In some embodiments, a majority of the outer surface 109 (shown in FIG. 1C) of the fluid permeable body 120 interfaces with an inner surface 103 (shown in FIG. 1C) of the fluid impermeable barrier 106. A singular fluid permeable body 120 may be advantageous to conventional systems, which typically require an air-laid nonwoven pad covered by a ribbed fabric compression bandage, because a singular fluid permeable body 120 reduced the number of components in the fluid collection device 100, reduces the assembly time of the fluid collection device 100, requires shelf-life data for only a single component, and provides a latex-free single component. In some embodiments, at least a portion of the singular porous material of the fluid permeable body 120 extends continuously between the opening 106 and the reservoir 122 to wick any fluid from the opening 106 directly to the reservoir 122. Moreover, as the fluid impermeable barrier 102 is flexible and the fluid permeable body 120 is configured to wick fluid from the body rather than absorb fluid from the body and hold the fluid against the body, the fluid collection device 100, in some embodiments, is free from a seal or cushioning ring on the inward edge 129 defining the opening 106. In these and other embodiments, the fluid permeable body 120 includes an outer surface and a single layer or type of material between the opening 106 and the conduit 108 positioned within the fluid permeable body 120.

In other embodiments, the fluid permeable body 120 may include two or more layers of fluid permeable materials and include no (or an absence of) more than two layers of material between the opening 106 and the conduit 108 positioned within the fluid permeable body 120. For example, the fluid collection device 100 may include a fluid permeable membrane covering or wrapping around at least a portion of a fluid permeable body, with both the fluid permeable membrane and the fluid permeable body being disposed in the chamber 104. The fluid permeable membrane may cover or extend across at least a portion (e.g., all) of the opening 106. The fluid permeable membrane may be configured to wick any fluid away from the opening 106, thereby preventing the fluid from escaping the chamber 104. The permeable properties referred to herein may be wicking, capillary action, diffusion, or other similar properties or processes, and are referred to herein as “permeable” and/or “wicking.” In some embodiments, at least one of the fluid permeable membrane or the fluid permeable support include nylon configured to wick fluid away from the opening 106. The material of the fluid permeable membrane and the fluid permeable support also may include natural fibers. In such examples, the material may have a coating to prevent or limit absorption of fluid into the material, such as a water repellent coating. Such “wicking” may not include absorption into the wicking material. Put another way, substantially no absorption of fluid into the material may take place after the material is exposed to the fluid and removed from the fluid for a time. While no absorption is desired, the term “substantially no absorption” may allow for nominal amounts of absorption of fluid into the wicking material (e.g., absorbency), such as less than about 10 wt % of the dry weight of the wicking material, less than about 7 wt %, less than about 5 wt %, less than about 3 wt %, less than about 2 wt %, less than about 1 wt %, or less than about 0.5 wt % of the dry weight of the wicking material.

The fluid permeable membrane may also wick the fluid generally towards an interior of the chamber 104, as discussed in more detail below. The fluid permeable membrane may include any material that may wick the fluid. For example, the fluid permeable membrane may include fabric, such as a gauze (e.g., a silk, linen, polymer based materials such as polyester, or cotton gauze), another soft fabric (e.g., jersey knit fabric or the like), or another smooth fabric (e.g., rayon, satin, or the like). Forming the fluid permeable membrane from gauze, soft fabric, and/or smooth fabric may reduce chaffing caused by the fluid collection device 100. Other embodiments of fluid permeable membranes, fluid permeable supports, chambers, and their shapes and configurations are disclosed in U.S. patent application Ser. No. 15/612,325 filed on Jun. 2, 2017; U.S. patent application Ser. No. 15/260,103 filed on Sep. 8, 2016; U.S. patent application Ser. No. 15/611,587 filed on Jun. 1, 2017; PCT Patent Application No. PCT/US19/29608, filed on Apr. 29, 2019, the disclosure of each of which is incorporated herein, in its entirety, by this reference. In many embodiments, the fluid permeable body 120 includes a fluid permeable support including a porous nylon structure (e.g., spun nylon fibers) and a fluid permeable membrane including gauze about or over the porous nylon structure.

FIG. 1B is a front view of a fluid collection device 100 in use on a female user 150. In use, the fluid permeable body 120 of the fluid collection device is positioned adjacent to a urethra of the user 150. The fluid permeable body 120 is disposed within a chamber 104 (shown in FIGS. 2A and 2B) of the fluid impermeable barrier 102 of the fluid collection device 100 and is exposed to the urethra of the user 150 through the opening 106 in the fluid collection device 100. The fluid collection device 100 may be secured to the user with any of a number of securing devices. Fluids received in the chamber 104 of the fluid collection device 100 from the urethra may be removed through the conduit 108.

FIG. 2A is a cross-sectional view of the fluid collection device 100 taken along line 2-2 of FIG. 1A. The fluid collection device 100 also includes conduit 108 that is at least partially disposed in the chamber 104. The conduit 108 (e.g., a tube) includes an inlet 110 at a second end region 127 of the fluid impermeable barrier 102 and an outlet 112 at a first end region 125 of the fluid impermeable barrier 102 positioned downstream from the inlet 110. The conduit 108 provides fluid communication between an interior region of the chamber 104 and a fluid storage container (not shown) or a portable vacuum source (not shown). For example, the conduit 108 may directly or indirectly fluidly couple the interior region of the chamber 104 and/or the reservoir 122 with the fluid storage container or the portable vacuum source.

In the illustrated embodiment, the fluid permeable body 120 defines a bore 202 extending through the fluid permeable body 120 from a first body end 121 of the fluid permeable body 120 to a second body end 123 of the fluid permeable body 120 distal to the first body end 120. In other embodiments, the bore 202 extends only partially into the fluid permeable body from the first body end 121 of the fluid permeable body 120.

In the illustrated embodiment, the conduit 108 is at least partially disposed in the chamber 104 and interfaces at least a portion of the bore 202 of the fluid permeable body 120. For example, the conduit 108 may extend into the fluid impermeable barrier 102 from the first end region 125 (e.g., proximate to the outlet 112) and may extend through the bore 202 to the second end region 127 (e.g., opposite the first end region 125) to a point proximate to the reservoir 122 such that the inlet 110 is in fluid communication with the reservoir 122. For example, in the illustrated embodiment, the inlet 110 is positioned in the reservoir 122. However, in other embodiments, the inlet 110 may be positioned flush with or behind an end of the fluid permeable body 120 that partially defines the reservoir 122. The fluid collected in the fluid collection device 100 may be removed from the interior region of the chamber 104 via the conduit 108. The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some embodiments, the conduit 108 may include silicone or latex.

The fluid impermeable barrier 102 may store fluids in the reservoir 122 therein. The reservoir 122 is an unoccupied portion of the chamber 104 and is void of other material. In some embodiments, the reservoir 122 is defined at least partially by the fluid permeable body 120 and the fluid impermeable barrier 102. For example, in an embodiment, the reservoir 122 may be located at the portion of the chamber 104 that is closest to the inlet 110 (e.g., the second end region). Accordingly, in the embodiment in FIG. 2A, the reservoir 122 is defined by the second body end 123 of the fluid permeable body 120 and the second end region 127 of the fluid impermeable barrier 122. However, the reservoir 122 may be located at different locations in the chamber 104. For example, the reservoir 122 may be located at the end of the chamber 104 that is closest to the outlet 112. In these and other embodiments, the conduit 108 may extend through the first end region 125 of the fluid impermeable barrier 102 and to the reservoir 122 without extending through the fluid permeable body 120. Accordingly, in these and other embodiments, the fluid permeable body 120 may be free from the bore. In another embodiment, the fluid collection device 100 may include multiple reservoirs, such as a first reservoir that is located at the portion of the chamber of the chamber 104 that is closest to the inlet 110 (e.g., second end region) and a second reservoir that is located at the portion of the of the chamber 104 that is closest to the outlet 112 (e.g., first end region). In another example, the fluid permeable body 120 is spaced from at least a portion of the conduit 108 and the reservoir 122 may be the space between the fluid permeable body 120 and the conduit 108. Other embodiments of reservoirs, fluid impermeable barriers, fluid permeable membranes, fluid permeable bodies, chambers, and their shapes and configurations are disclosed in U.S. patent application Ser. No. 15/612,325 filed on Jun. 2, 2017; U.S. patent application Ser. No. 15/260,103 filed on Sep. 8, 2016; and U.S. patent application Ser. No. 15/611,587 filed on Jun. 1, 2017, the disclosure of each of which is incorporated herein, in its entirety, by this reference.

The fluid impermeable barrier 102 and the fluid permeable body 120 may be configured to have the conduit 108 at least partially disposed in the chamber 104. For example, the fluid permeable body 120 may be configured to form a space that accommodates the conduit 108, such as the bore 202. In another example, the fluid impermeable barrier 102 may define an aperture 124 sized to receive the conduit 108 (e.g., at least one tube). The at least one conduit 108 may be disposed in the chamber 104 via the aperture 124. The apertures 124 may be configured to form an at least substantially fluid tight seal against the conduit 108 or the at least one tube thereby substantially preventing the fluids from escaping the chamber 104.

In some embodiments, the conduit 108 may extend through the fluid permeable body 120 and at least partially into the reservoir 122, as shown in FIG. 2A. In some embodiments, the conduit 108 may extend through the fluid permeable body 120 and terminate at or before the second body end 123 of the fluid permeable body 120 such that the conduit 108 does not extend into the reservoir 122 (or the reservoir 122 is absent of the conduit 108). For example, as shown in FIG. 2B, an end of the conduit 108 may be generally flush or coplanar with the second body end 123 of the fluid permeable body 120. In other embodiments, the end of the conduit 108 may be recessed from the second body end 123 of the fluid permeable body 120. The end of the conduit 108 also may be selectively moveable between partially extending into the reservoir 122 (shown in FIG. 2A) and recessed from or flush with the second body end 123 of the fluid permeable body (shown in FIG. 2B).

When secured to the fluid collection device 100, the conduit 108 is configured to provide fluid communication with and at least partially extend between one or more of a fluid storage containers (not shown) and a portable vacuum source (not shown). For example, the conduit 108 may be configured to be fluidly coupled to and at least partially extend between one or more of the fluid storage containers and the portable vacuum source. In an embodiment, the conduit 108 is configured to be directly connected to the portable vacuum source (not shown). In such an example, the conduit 108 may extend from the fluid impermeable barrier 102 by at least one foot, at least two feet, at least three feet, or at least six feet. In another example, the conduit 108 is configured to be indirectly connected to at least one of the fluid storage container (not shown) or the portable vacuum source (not shown). In some examples, the conduit may be frosted or opaque (e.g., black) to obscure visibility of the fluids therein. In some embodiments, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

The inlet 110 and the outlet 112 are configured to provide fluid communication (e.g., directly or indirectly) between the portable vacuum source (not shown) and the chamber 104 (e.g., the reservoir 122). For example, the inlet 110 and the outlet 112 of the conduit 108 may be configured to directly or indirectly fluidly couple the portable vacuum source to the reservoir 122. In an embodiment, the inlet 110 and/or the outlet 112 may form a male connector. In another example, the inlet 110 and/or the outlet 112 may form a female connector. In an embodiment, the inlet 110 and/or the outlet 112 may include ribs that are configured to facilitate secure couplings. In an embodiment, the inlet 110 and/or the outlet 112 may form a tapered shape. In an embodiment, the inlet 110 and/or the outlet 112 may include a rigid or flexible material.

Locating the inlet 110 at or near a gravimetrically low point of the chamber 104 enables the conduit to receive more of the fluids than if inlet 110 was located elsewhere and reduce the likelihood of pooling (e.g., pooling of the fluids may cause microbe growth and foul odors). For instance, the fluids in the fluid permeable body 120 may flow in any direction due to capillary forces. However, the fluids may exhibit a preference to flow in the direction of gravity, especially when at least a portion of the fluid permeable body 120 is saturated with the fluids.

As the portable vacuum source applies a vacuum/suction in the conduit 108, the fluid(s) in the chamber 104 (e.g., such as in the reservoir 122 positioned at the first end region 125, the second end region 127, or other intermediary positions within the chamber 104) may be drawn into the inlet 110 and out of the fluid collection device 100 via the conduit 108.

In an embodiment, the conduit 108 is configured to be at least insertable into the chamber 104. In such an embodiment, the conduit 108 may include one or more markers 131 (shown in FIG. 1A) on an exterior thereof that are configured to facilitate insertion of the conduit 108 into the chamber 104. For example, the conduit 108 may include one or more markings thereon that are configured to prevent over or under insertion of the conduit 108, such as when the conduit 108 defines an inlet 110 that is configured to be disposed in or adjacent to the reservoir 122. In another embodiment, the conduit 108 may include one or more markings thereon that are configured to facilitate correct rotation of the conduit 108 relative to the chamber 104. In an embodiment, the one or more markings may include a line, a dot, a sticker, or any other suitable marking. In examples, the conduit 108 may extend into the fluid impermeable barrier 102 from the first end region (e.g., proximate to the outlet 112) and may extend to the second end region (e.g., opposite the first end region) to a point proximate to the reservoir 122 such that the inlet 110 is in fluid communication with the reservoir 122. In some embodiments (not shown), the conduit 108 may enter the second end region and the inlet 110 may be disposed in the second end region (e.g., in the reservoir 122). The fluid collected in the fluid collection device 100 may be removed from the interior region of the chamber 104 via the conduit 108. The conduit 108 may include a flexible material such as plastic tubing (e.g., medical tubing) as disclosed herein. In some examples, the conduit 108 may include one or more portions that are resilient, such as to by having one or more of a diameter or wall thickness that allows the conduit to be flexible.

In an embodiment, one or more components of the fluid collection device 100 may include an antimicrobial material, such as an antibacterial material where the fluid collection device may contact the wearer or the bodily fluid of the wearer. The antimicrobial material may include an antimicrobial coating, such as a nitrofurazone or silver coating. The antimicrobial material may inhibit microbial growth, such as microbial growth due to pooling or stagnation of the fluids. In an embodiment, one or more components of the fluid collection device 100 (e.g., impermeable barrier 102, conduit 108, etc.) may include an odor blocking or absorbing material such as a cyclodextrine containing material or a thermoplastic elastomer (TPE) polymer.

In any of the embodiments disclosed herein, the conduits 108 may include or be operably coupled to a flow meter (not shown) to measure the flow of fluids therein, one or more securement devices (e.g., a StatLock securement device, not shown) or fittings to secure the conduit 108 to one or more components of the systems or devices disclosed herein (e.g., portable vacuum source or fluid storage container), or one or more valves to control the flow of fluids in the systems and devices herein. In an embodiment, at least one of portion of the conduit 108 of the fluid collection devices or systems herein may be formed of an at least partially opaque material which may obscure the fluids that are present therein. For example, a first section of the conduit 108 disclosed herein may be formed of an opaque material or translucent material while a second section of the conduit 108 may be formed of a transparent material or translucent material. In some embodiments, the first section may include transparent or translucent material. Unlike the opaque or nearly opaque material, the translucent material allows a user of the devices and systems herein to visually identify fluids or issues that are inhibiting the flow of fluids within the conduit 108.

In any of the examples, systems or devices disclosed herein, the system of fluid collection device may include moisture sensors (not shown) disposed inside of the chamber of the fluid collection device. In such examples, the moisture sensor may be operably coupled to a controller or directly to the portable vacuum source, and may provide electrical signals indicating that moisture is or is not detected in one or more portions of the chamber. The moisture sensor(s) may provide an indication that moisture is present, and responsive thereto, the controller or portable vacuum device may direct the initiation of suction to the chamber to remove the fluid therefrom. Suitable moisture sensors may include capacitance sensors, volumetric sensors, potential sensors, resistance sensors, frequency domain reflectometry sensors, time domain reflectometry sensors, or any other suitable moisture sensor. In practice, the moisture sensors may detect moisture in the chamber and may provide a signal to the controller or portable vacuum source to activate the portable suction device.

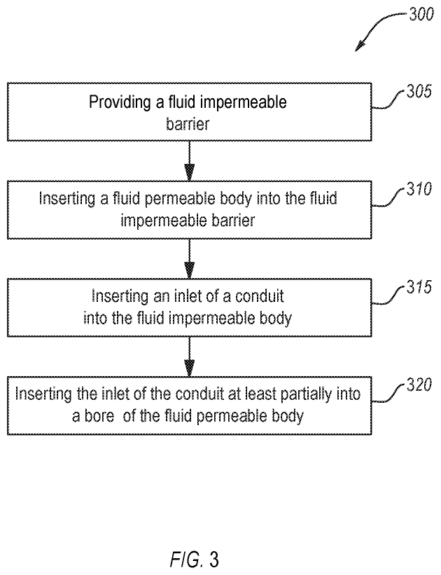

FIG. 3 is a flow diagram of a method 300 of assembling the fluid collection devices and/or fluid collection systems disclosed herein, according to an embodiment. The method 300 may include act 305, which recites providing a fluid impermeable barrier. The fluid impermeable barrier at least partially defines a chamber and also an opening extending therethrough. The opening is configured to be positioned adjacent to a female urethra or have a male urethra positioned therethrough. The fluid permeable body may include a singular porous hydrophilic polyolefin material extruded, molded, or sintered to a substantially cylindrical shape

The method may include act 310, which recites inserting a substantially cylindrical and fluid permeable body into the chamber of the fluid impermeable barrier. When the fluid permeable body is inserted into the chamber of the fluid impermeable barrier, the fluid permeable body interfaces at least a portion of the fluid impermeable barrier and covers at least a portion of the opening. The fluid permeable body includes a singular porous material that is substantially cylindrical in shape and configured to wick any fluid away from the opening. In some embodiments, act 310 may include inserting the fluid permeable body into the chamber of the fluid impermeable barrier such that a reservoir is defined within the chamber by a second body end of the fluid permeable body distal to the first body end and a second end region of the fluid impermeable barrier distal to the aperture. In some embodiments, act 310 may include inserting the substantially cylindrical and fluid permeable body into the chamber of the fluid impermeable barrier such that the fluid permeable body and the conduit fill substantially all of the chamber.

The method may include act 315, which recites inserting an inlet of a conduit into the fluid impermeable body. The conduit may be inserted into the fluid impermeable body through an aperture defined by the fluid impermeable barrier at a first end region of the fluid impermeable barrier. In some embodiments, act 315 may include inserting the inlet of the conduit into the bore at the first body end, through the bore of the fluid permeable body, through the second body end of the fluid permeable body, and into the reservoir such that the conduit extends from the reservoir, through the fluid permeable body, through the aperture to outside the fluid impermeable barrier.

The method may include an act 320, which recites inserting the inlet of the conduit at least partially into a bore at a first body end of the fluid permeable body. The bore extends at least partially through the fluid permeable body and is defined by the fluid permeable body. The conduit interfaces at least a portion of the fluid permeable body.

Acts 305, 310, 315, and 320 of the method 300 are for illustrative purposes. For example, the act 305, 310, 315, and 320 of the method 300 may be performed in different orders, split into multiple acts, modified, supplemented, or combined. In an embodiment, one or more of the acts 305, 310, 315, and 320 of the method 300 may be omitted from the method 300. Any of the acts 305, 310, 315, and 320 may include using any of the fluid collection devices or systems disclosed herein.

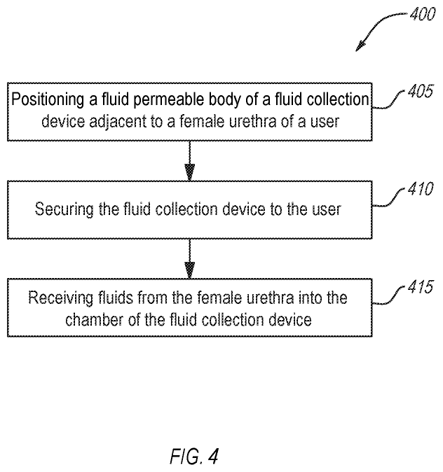

FIG. 4 is a flow diagram of a method 400 for collecting fluids. The method 400 includes an act 405 of positioning a fluid permeable body of a fluid collection device adjacent to a female urethra of a user. The fluid permeable body is disposed within a chamber of a fluid impermeable barrier of the fluid collection device and exposed to the female urethra of the user through an opening in the fluid collection device defined by the fluid impermeable barrier. The method 400 also includes an act 410 of securing the fluid collection device to the user. The method 400 also includes an act 415 of receiving fluids from the female urethra into the chamber of the fluid collection device. In some embodiments, the method 400 an act of applying suction effective to suction the fluids from the chamber via a conduit disposed therein.

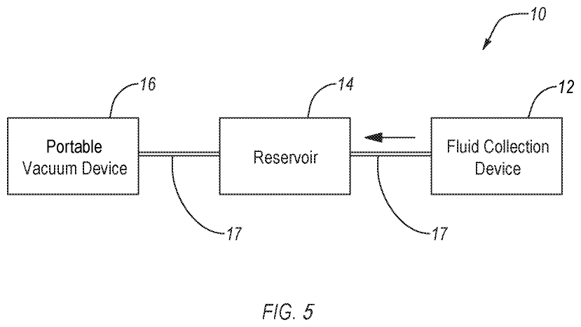

FIG. 5 is a block diagram of a system 10 for fluid collection, according to an embodiment. The system 10 includes a fluid collection device 12, a fluid storage container 14, and a portable vacuum source 16. The fluid collection device 12 may include any of the fluid collection devices described herein, such as the fluid collection device 100. The fluid collection device 12, the fluid storage container 14, and the portable vacuum source 16 may be fluidly coupled to each other via one or more conduits 17. The conduit 17 may include any of the conduits described herein, such as the conduit 108. The fluid collection device 12 may be operably coupled to one or more of the fluid storage container 14 or the portable vacuum source via the conduit 17. Fluid (e.g., urine or other bodily fluids) collected in the fluid collection device 12 may be removed from the fluid collection device 12 via the conduit 17, which protrudes into an interior region of the fluid collection device 12. For example, a first open end of the conduit 17 may extend into the fluid collection device 12 to a reservoir therein. The second open end of the conduit 17 may extend into the fluid collection device 12 or the portable vacuum source 16. The suction force may be introduced into the interior region of the fluid collection device 12 via the first open end of the conduit 17 responsive to a suction (e.g., vacuum) force applied at the second end of the conduit 17. The suction force may be applied to the second open end of the conduit 17 by the portable vacuum source 16 either directly or indirectly.

The suction force may be applied indirectly via the fluid storage container 14. For example, the second open end of the conduit 17 may be disposed within the fluid storage container 14 and an additional conduit 17 may extend from the fluid storage container 14 to the portable vacuum source 16. Accordingly, the portable vacuum source 16 may apply suction to the fluid collection device 12 via the fluid storage container 14. The suction force may be applied directly via the fluid storage container 14. For example, the second open end of the conduit 17 may be disposed within the portable vacuum source 16. An additional conduit 17 may extend from the portable vacuum source 16 to a point outside of the fluid collection device 12, such as to the fluid storage container 14. In such examples, the portable vacuum source 16 may be disposed between the fluid collection device 12 and the fluid storage container 14.

The fluid collection device 12 may be shaped and sized to be positioned adjacent to a female urethra. The fluid collection member of the fluid collection device 12 may include a fluid impermeable barrier at least partially defining a chamber (e.g., interior region of the fluid collection device member) of the fluid collection device 12. As described in more detail above, the fluid collection device 12 may include a softer, thinner fluid impermeable barrier than conventional fluid collection devices. The fluid impermeable barrier also defines an opening extending therethrough from the external environment. The opening may be positioned on the fluid collection member to be aligned adjacent to a female urethra. The fluid collection member of the fluid collection device 12 may include a fluid permeable body disposed within the fluid impermeable barrier. The fluid permeably body may include a fluid permeable membrane and fluid permeable support disposed within the fluid permeable membrane. The conduit 17 may extend into the fluid collection device 12 at a first end region, through one or more of the fluid impermeable barrier, fluid permeable membrane, or the fluid permeable support to a second end region of the fluid collection member of the fluid collection device 12. Example fluid collection devices for use with the systems and methods herein are described in more detail below.

In some embodiments, the fluid storage container 14 may include a bag (e.g., drainage bag), a bottle or cup (e.g., collection jar), or any other enclosed container for storing bodily fluids such as urine. In examples, the conduit 17 may extend from the fluid collection device 12 and attach to the fluid storage container 14 at a first point therein. An additional conduit 17 may attach to the fluid storage container 14 at a second point thereon and may extend and attach to the portable vacuum source 16. For example, the fluid storage container 14 may include a container fluidly coupled to a first conduit section that is also fluidly coupled to the fluid collection member of the fluid collection device 12. The container may be fluidly coupled to a second section of the conduit 17 that is also fluidly coupled to a portable vacuum source. In such examples, the portable vacuum source 16 may provide a vacuum/suction through the container to the fluid collection member to provide suction in the chamber of the fluid collection member. Accordingly, a vacuum (e.g., suction) may be drawn through fluid collection device 12 via the fluid storage container 14. As the fluid is drained from the chamber, the fluid may travel through the first section of conduit to the fluid storage container where it may be retained. Fluid, such as urine, may be drained from the fluid collection device 12 using the portable vacuum source 16.

In some embodiments, the portable vacuum source 16 may be disposed in or on the fluid collection device 12. In such examples, the conduit 17 may extend from the fluid collection device and attach to the portable vacuum source 16 at a first point therein. An additional conduit 17 may attach to the portable vacuum source 16 at a second point thereon and may extend out of the fluid collection device 12, and may attach to the fluid storage container 14. Accordingly, a vacuum (e.g., suction) may be drawn through fluid collection device 12 via the fluid storage container 14.

The portable vacuum source 16 may include one or more of a manual vacuum pump, and electric vacuum pump, a diaphragm pump, a centrifugal pump, a displacement pump, a magnetically driven pump, a peristaltic pump, or any pump configured to produce a vacuum. The portable vacuum source 16 may provide a vacuum or suction to remove fluid from the fluid collection member of the fluid collection device 12. In some embodiments, the portable vacuum source 16 may be powered by one or more of a power cord (e.g., connected to a power socket), one or more batteries, or even manual power (e.g., a hand operated vacuum pump). In examples, the portable vacuum source 16 may be sized and shaped to fit outside of, on, or within the fluid collection device 12. For example, the portable vacuum source 16 may include one or more miniaturized pumps or one or more micro pumps. The portable vacuum sources 16 disclosed herein may include one or more of a switch, a button, a plug, a remote, or any other device suitable to activate the portable vacuum source 16. It should be understood that the portable vacuum sources 16 disclosed herein may provide a portable means of providing a suction or vacuum that allows use of the devices and systems herein outside of hospital or care facility environments where vacuum lines are plumbed into patient rooms or large (e.g., larger or heavier than a patient can readily carry) vacuum sources are located. For example, a portable vacuum source may be small and light enough to be carried by a user (e.g., patient) or aid (e.g., nurse) during transportation of the user.

As used herein, the term “about” or “substantially” refers to an allowable variance of the term modified by “about” by ±10% or ±5%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiment disclosed herein are for purposes of illustration and are not intended to be limiting.

1. A fluid collection device, comprising:
a fluid impermeable barrier having a shore A durometer hardness of less than about 20 and at least partially defining a chamber, an opening extending longitudinally along the fluid impermeable barrier and configured to be positioned adjacent to a female urethra, and an aperture configured to receive a conduit therethrough; and
a fluid permeable body positioned at least partially within the chamber to extend across at least a portion of the opening and configured to wick fluid away from the opening.

2. The fluid collection device of claim 1, wherein the fluid impermeable barrier has a thickness of about 0.2 mm to about 3.2 mm.

3. The fluid collection device of claim 1, wherein the fluid impermeable barrier has a thickness of less than about 3.2 mm.

4. The fluid collection device of claim 1, wherein the fluid impermeable barrier has a thickness of less than about 1.6 mm.

5. The fluid collection device of claim 1, wherein the fluid impermeable barrier has a thickness of less than about 0.8 mm.

6. The fluid collection device of claim 1, wherein the fluid impermeable barrier includes a polyurethane film.

7. The fluid collection device of claim 1, further comprising a conduit including an inlet and an outlet, wherein at least a portion of the conduit extends through the aperture and into the chamber, fluid permeable body being configured to wick fluid away from the opening to the inlet.

8. The fluid collection device of claim 7, wherein:
the fluid permeable body includes an outer surface and no more than two layers of material between the opening and the conduit; and
the fluid impermeable barrier includes an inner surface at least partially defining the chamber, the inner surface interfacing the outer surface of the fluid permeable body.

9. The fluid collection device of claim 1, wherein the fluid impermeable barrier includes a modulus of elasticity of about 4 N/in to about 20 N/in.

10. The fluid collection device of claim 1, wherein the fluid impermeable barrier has a shore A durometer of hardness of less than 10.

11. The fluid collection device of claim 1, wherein the fluid impermeable barrier has a shore A durometer of hardness of less than 5.

12. A fluid collection device, comprising:
a fluid impermeable barrier having a thickness of about 0.2 mm to about 3.2 mm and at least partially defining a chamber, an opening extending longitudinally along the fluid impermeable barrier and configured to be positioned adjacent to a female urethra, and an aperture configured to receive a conduit therethrough; and
a fluid permeable body positioned at least partially within the chamber to extend across at least a portion of the opening and configured to wick fluid away from the opening.

13. The fluid collection device of claim 12, wherein the fluid impermeable barrier has a modulus of elasticity of about 4 N/in to about 20 N/in.

14. The fluid collection device of claim 12, wherein the fluid impermeable barrier has a thickness of about 0.2 mm to about 1.6 mm.

15. The fluid collection device of claim 12, wherein the fluid impermeable barrier has a thickness of about 0.2 mm to about 0.8 mm.

16. The fluid collection device of claim 12, wherein the fluid impermeable barrier includes a polyurethane film.

17. The fluid collection device of claim 12, further comprising a conduit including an inlet and an outlet, wherein at least a portion of the conduit extends through the aperture and into the chamber, fluid permeable body being configured to wick fluid away from the opening to the inlet.

18. The fluid collection device of claim 17, wherein:
the fluid permeable body includes an outer surface and no more than two layers of material between the opening and the conduit; and
the fluid impermeable barrier includes an inner surface at least partially defining the chamber, the inner surface interfacing the outer surface of the fluid permeable body.

19. The fluid collection device of claim 12, wherein the fluid impermeable barrier includes a shore A durometer hardness of less than about 15.

20. The fluid collection device of claim 12, wherein the fluid impermeable barrier includes a shore A durometer hardness of less than about 10.

21. The fluid collection device of claim 12, wherein the fluid impermeable barrier includes a shore A durometer hardness of less than about 5.

22. A fluid collection device, comprising:
a fluid impermeable barrier having a modulus of elasticity of about 4 N/in to about 20 N/in and at least partially defining a chamber, an opening extending longitudinally along the fluid impermeable barrier and configured to be positioned adjacent to a female urethra, and an aperture configured to receive a conduit therethrough; and
a fluid permeable body positioned at least partially within the chamber to extend across at least a portion of the opening and configured to wick fluid away from the opening.

23. The fluid collection device of claim 22, wherein the fluid impermeable barrier has a thickness of about 0.2 mm to about 3.2 mm.

24. The fluid collection device of claim 22, wherein the fluid impermeable barrier has a thickness of about 0.2 mm to about 1.6 mm.

25. The fluid collection device of claim 22, wherein the fluid impermeable barrier has a thickness of about 0.2 mm to about 0.8 mm.

26. The fluid collection device of claim 22, wherein the fluid impermeable barrier includes a polyurethane film.

27. The fluid collection device of claim 22, further comprising a conduit including an inlet and an outlet, wherein at least a portion of the conduit extends through the aperture and into the chamber, fluid permeable body being configured to wick fluid away from the opening to the inlet.

28. The fluid collection device of claim 27, wherein:
the fluid permeable body includes an outer surface and no more than two layers of material between the opening and the conduit; and
the fluid impermeable barrier includes an inner surface at least partially defining the chamber, the inner surface interfacing the outer surface of the fluid permeable body.

29. The fluid collection device of claim 22, wherein the fluid impermeable barrier includes a shore A durometer hardness of less than about 15.

30. The fluid collection device of claim 22, wherein the fluid impermeable barrier includes a shore A durometer hardness of less than about 10.

31. The fluid collection device of claim 22, wherein the fluid impermeable barrier includes a shore A durometer hardness of less than about 5.
